(19) United States
(12) Patent Application Publication
(10) Pub. No.: US 2024/0423832 A1
(43) Pub. Date: Dec. 26, 2024

(54) SURGICAL FIXATION TOOL WITH COUPLING TO INJECTOR/SYRINGE

(71) Applicant: Everads Therapy Ltd., Tel Aviv (IL)

(72) Inventors: Hagay Drori, Tel Aviv (IL); Gad Lewkonya, Neve-Miftach (IL); David Daily, Herzliya (IL); Lior Raday, Bror Hayil (IL)

(73) Assignee: Everads Therapy Ltd., Tel Aviv (IL)

(21) Appl. No.: 18/684,732

(22) PCT Filed: Aug. 22, 2022
(86) PCT No.: PCT/IB2022/057830

§ 371 Date: Feb. 19, 2024

Related U.S. Application Data
(60) Provisional application No. 63/238,875, filed on Aug. 31, 2021.

(51) Int. Cl. A61F 9/00

(52) U.S. Cl. CPC A61F 9/0017

(57) ABSTRACT

A kit includes an injector (1) including a substance syringe, which includes a plunger (3) that slides in a lumen of a barrel (4) which is coupled to a lumen of a needle (5), a fixation tool (10), and a coupling assembly (60) that includes a fixation-tool force transfer device (62) coupled to the fixation tool (10). The fixation-tool force transfer device (62) is coupled to the plunger (4) of the injector (1) such that actuation of the fixation-tool force transfer device (61) moves the plunger (4) of the injector (!).

### FIELD OF THE INVENTION

The present invention is related generally to surgical devices and particularly to a surgical fixation tool for use in procedures, such as in ophthalmology, which is coupled to an injector or syringe.

### BACKGROUND OF THE INVENTION

PCT Application WO 2019/202603 describes a device, inter alia, for injection of a medication into an interlayer, such as the suprachoroidal layer of the eye. The injector 1 of the device is shown in FIG. 1. The injector 1 includes a substance syringe 2, which has a plunger 3 that slides in a lumen of a barrel 4. Plunger 3 is arranged to deliver a substance stored in a distal portion of the lumen of barrel 4 into a needle 5. The term “plunger” throughout includes both the rod (plunger rod) and the piston member that actually pushes against the substance.

Needle 5 has a sharp distal tip, which in the illustration is covered by a needle guard 6. The injector 1 also includes an elongated tissue separator 7 that is arranged to pass through the lumen of needle 5. (The substance can flow around the tissue separator 7 to pass through the needle tip.) An actuator 8 linearly moves the tissue separator 7 in the needle lumen between a first position, in which the separator distal tip is fixedly positioned proximally to the needle distal tip, and a second position in which the separator distal tip is fixedly positioned a predetermined distance distally from the needle distal tip.

Needle 5 is configured to penetrate into and/or through a target portion of an outer layer of the organ, such as the sclera of an eye (optionally including also the conjunctiva). Tissue separator 7 is configured for passing through needle 5 and can be positioned at different positions in accordance with different functions, including at least one of: (i) forming a directional spaced passage, by entering and separating (spacing-apart) adjacent layers in an interlayer region portion, (ii) assist in restricting penetration length of needle 5 in the target tissue portion, and (iii) channeling a fluid from syringe 2 into the lumen of needle 5. A sealed connector member 9 may be used to provide a fluid tight connection between tissue separator 7 and needle 5.

### SUMMARY

The present invention seeks to provide an improved surgical fixation tool for use in surgical procedures, such as ophthalmic procedures, such as but not limited to, injection of a substance into an interlayer, such as the suprachoroidal layer of the eye.

For use in ophthalmology, the fixation tool may be used to manipulate, position and/or stabilize the eye. For example, the fixation tool may be used as a counterforce against forces applied during a surgical procedure, injection, diagnostic procedure, etc. The fixation tool may also be used to rotate or translate the ocular tissue during a surgical procedure.

The fixation tool may be used to stretch and hold the conjunctiva to allow for easy penetration into/under the sclera. Likewise, the fixation tool may be used to stretch and hold the sclera for easy penetration into/under the sclera by surgical tools (needle, knife or other tools). The fixation tool may be used to shift the orientation of the eyeball to ensure comfortable and optimal access for ophthalmic procedures. The fixation tool allows all of these procedures to be done atraumatically without perforating the sclera or other sensitive layers or tissues.

There is provided in accordance with a non-limiting embodiment of the invention a kit including an injector including a substance syringe, which includes a plunger that slides in a lumen of a barrel which is coupled to a lumen of a needle, a fixation tool including at least one fixation member which protrudes from a leg of a proximal grasping portion, the at least one fixation member including a distal tip, and a coupling assembly that includes a fixation-tool force transfer device, force transfer device may be a syringe having a plunger arranged to slide in a barrel, the fixation tool force transfer device including an attachment member extending therefrom that is coupled to the fixation tool, wherein the fixation tool force transfer device is fluidly coupled to an injector force transfer device; an injector force transfer device may by a syringe including an attachment member extending therefrom that is coupled to the plunger of the substance syringe such that actuation of the fixation tool force transfer device moves the plunger of the injector force transfer device and the plunger of the substance syringe within the injector.

### BRIEF DESCRIPTION OF THE DRAWINGS

The present invention will be better understood with reference to the following drawings and description. The components in the figures are not necessarily to scale, emphasis instead being placed upon illustrating the principles of the invention.

FIG. 1 is a simplified pictorial illustration of an injector of the prior art for injecting a substance into an eye, such as into the suprachoroidal space, and for creating a suprachoroidal passage for introducing the substance therein;

FIG. 2 is a simplified planar-view illustration of a fixation tool, constructed and operative in accordance with a non-limiting embodiment of the invention;

FIG. 3 is a simplified pictorial illustration of a coupling assembly for coupling the fixation tool of FIG. 2 with the injector of FIG. 1, so that a single user can grasp both instruments (the fixation tool and the injector) and also cause movement of the substance syringe plunger of the injector to deliver a substance from the injector;

FIG. 3A is a simplified exploded illustration of the fixation-tool force transfer device;

FIG. 3B is a simplified exploded illustration of the force transfer device; and

FIGS. 4A-6C are simplified pictorial illustrations of the progression of the movement of the plunger of the fixation tool force transfer device, which applies pressure on the plunger of the intermediate syringe, which causes movement of the plunger of the substance syringe.

### DETAILED DESCRIPTION

Reference is now made to FIG. 2, which illustrates a surgical fixation tool 10, constructed and operative in accordance with a non-limiting embodiment of the invention.

The surgical fixation tool 10 includes at least one fixation member 12. Although the invention can be carried out with just one fixation member 12, in the illustrated and preferred embodiment there are more than one fixation members 12, such as a pair of fixation members 12, each of which protrudes from a leg 16. Accordingly in the illustrated embodiment, there are a pair of legs 16 separated by a (e.g., U-shaped) channel 18. The legs 16 extend from a proximal grasping portion 20 of the tool 10. Each fixation member 12 includes a needle 28 with a sharp distal tip. The grasping portion 20 may extend from a shaft 21.

Throughout the disclosure, “proximal” is the direction, which in use of the tool is further away from the eye tissue (generally towards the user of the tool), and “distal” is the direction, which in use of the tool is towards the eye tissue or further into the eye tissue.

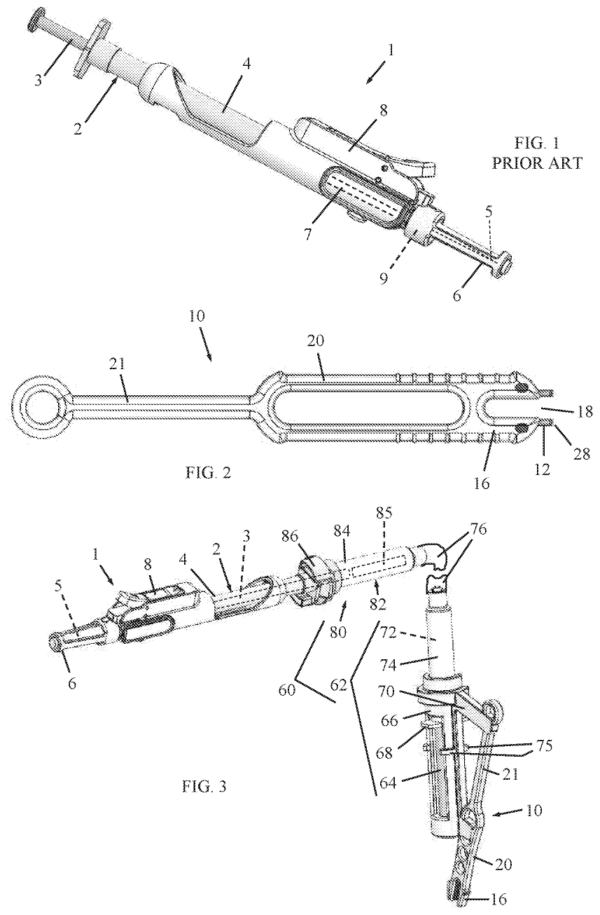

The fixation tool 10 may be used in surgical procedures, such as ophthalmic procedures, such as but not limited to, injection of a substance into an interlayer, such as the suprachoroidal layer of the eye, such as in the procedure described in PCT Application WO 2019/202603, or any other ophthalmic procedure that requires scleral or conjunctival fixation. The fixation tool 10 can fix the conjunctiva so that the injector 1 (FIG. 1) can create a suprachoroidal tunnel between the choroid and the sclera. The fixation tool 10 can apply counter forces to the multiple forces of the injector 1, which are in varying directions during the ophthalmic procedure.

However, a problem exists in using the injector 1 with the fixation tool 10. One hand of the surgeon must grasp the injector 1 close to the needle side (distal side) in order to maintain the injector 1 in a stable position and orientation. This means that same hand cannot reach and push the substance syringe plunger 3 at the proximal side of the injector 1 in order to deliver the substance to the space created by the tissue separator 7. The other hand of the surgeon is used to hold fixation tool 10 to fixate the ocular tissue. Thus, with this set of instruments, a third hand is needed to push the syringe plunger 3 at the proximal side of the injector 1 in order to deliver the substance.

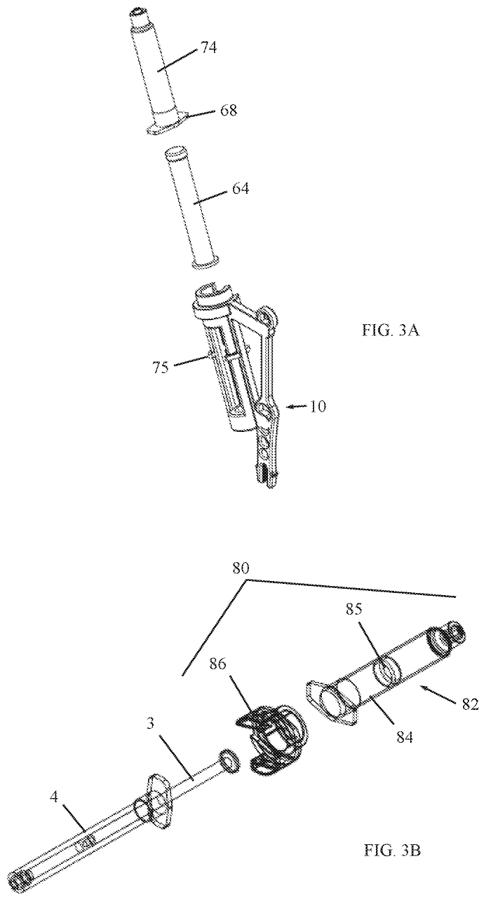

Reference is now made to FIG. 3, which illustrates a coupling assembly 60 for coupling the fixation tool 10 of FIG. 2 with the injector 1 of FIG. 1, so that a single user can grasp both instruments (the fixation tool and the injector) and also cause movement of the substance syringe plunger 3 of the injector 1 to deliver a substance from the injector 1.

It is noted that the invention is not limited to the fixation tool 10 of FIG. 2; the invention can be carried out with other fixation tools, such as but not limited to, forceps, tweezers, pincers, probes, markers, calipers, gauges, and other grasping, measurement or fixation tools, all of which are referred to as “fixation tools”.

Coupling assembly 60 may include a fixation-tool force transfer device 62 (refer also to FIG. 3A). In the non-limiting illustrated embodiment, the fixation-tool force transfer device 62 is a syringe which may include a plunger 64 arranged to slide in a barrel 74. Plunger 64 may include a user interface member 68, such as a ring or flanges coupled to plunger 64 which can easily be pushed or otherwise moved by the user's finger. The fixation-tool force transfer device 62 may include an attachment member 70 extending therefrom that couples to fixation tool 10, such as to shaft 21 or any other portion of fixation tool 10. In the illustrated embodiment, fixation-tool syringe force transfer device 62 is not parallel to the shaft 21 of fixation tool 10, which provides a better ergonomic angle for the user to hold fixation tool 10 and move user interface member 68. The angle between fixation-tool force transfer device 62 and shaft 21 of fixation tool 10 may range between 0-90°, and may be adjustable or fixed.

The fixation-tool force transfer device may be hydraulic, pneumatic, mechanical, electrical or any other kind of device for transferring a force to operate the injector 1.

Plunger 64 is arranged to press against a material 72, i.c., liquid (for hydraulic actuation) or gas (for pneumatic actuation), or a mechanical cable, stored in a barrel 74, which is fixated in a socket 66.

Coupling assembly 60 may include a connection 76, such as tubing, sealingly connected at one end thereof to an exit of barrel 74 and at an opposite end thereof to a force transfer device 80, which is coupled to the plunger 3 of syringe 2 of injector 1. In one non-limiting embodiment, force transfer device 80 includes an intermediate syringe 82, which has a barrel 84 that can accept the plunger 3 (that is, its proximal portion—plunger rod). A connector 86 may couple syringe 2 of injector 1 to the intermediate syringe 82. The intermediate syringe 82 may have a plunger 85. (Refer also to FIG. 3B.)

Accordingly, the proximal portion of plunger (plunger rod) 3 of the substance syringe 2 may be disposed in the barrel 84 of the intermediate syringe 82. The plunger 85 of the intermediate syringe 82 can contact and apply a force and movement on the plunger (plunger rod) 3 of the substance syringe 2 when pressure (e.g., hydraulic, pneumatic or mechanical pressure) is applied on the plunger 85 of the intermediate syringe 82 by movement of the plunger 64 of the fixation tool force transfer device 62. (In the case of an electrical fixation-tool force transfer device, plunger 85 of the intermediate syringe 82 may be moved by an electrical motor or other actuator.)

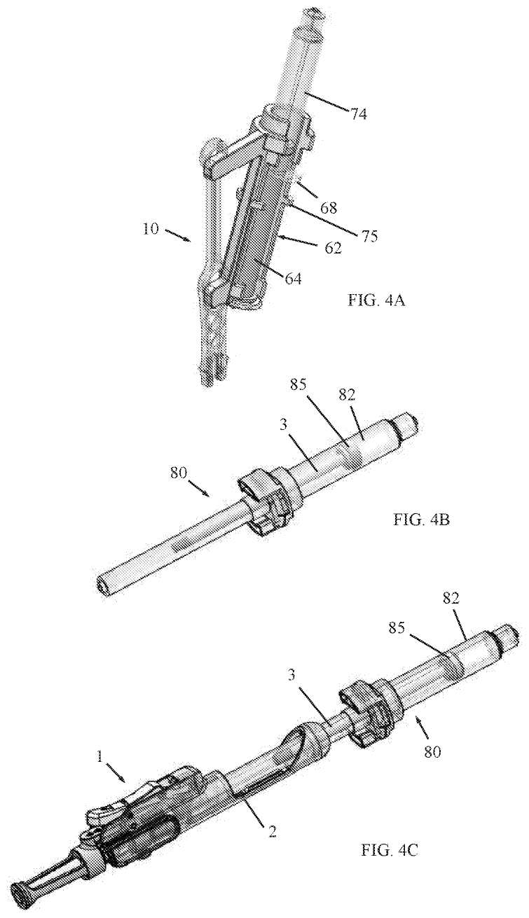

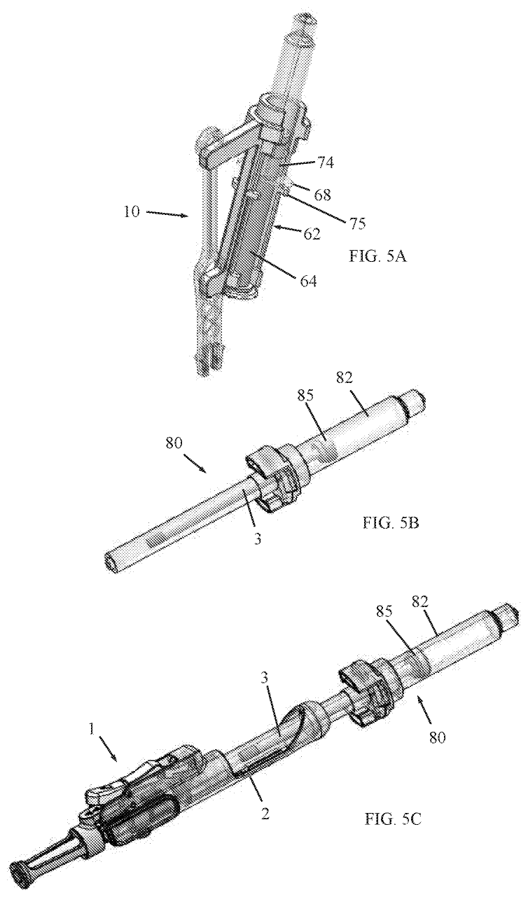

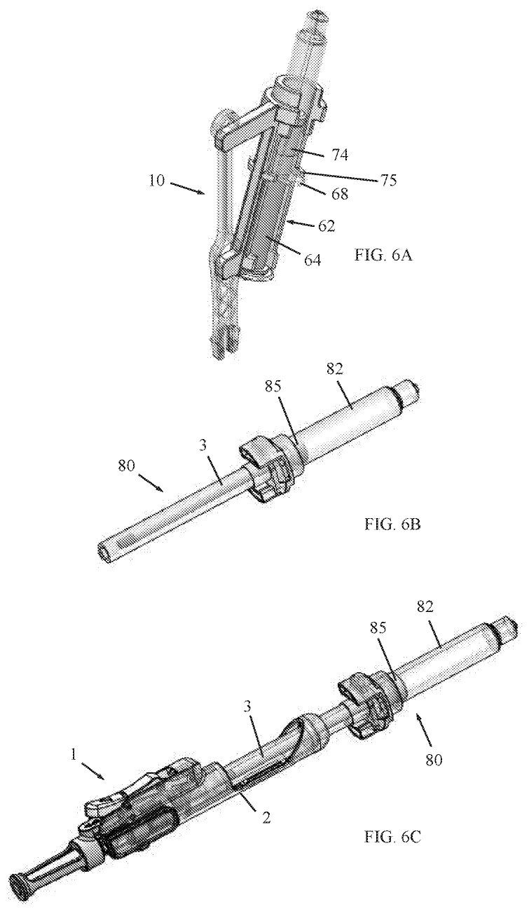

FIGS. 4A-6C illustrate the progression of the movement of the barrel 74 of the fixation tool force transfer device 62, which applies pressure on the plunger 85 of the intermediate syringe 82, which causes movement of the plunger 3 of the substance syringe 2. In FIGS. 4A, 5A, and 6A, the user interface member 68 is progressively moved downward, which progressively moves barrel 74 of fixation tool force transfer device 62, starting from before causing injection (FIG. 4A), in the middle of causing injection (FIG. 5A), and the end of causing injection (FIG. 6A), respectively.

(It is noted that optionally, as an opposite alternative, the barrel 74 may be fixed to the fixation tool 10, and the plunger 64 may move instead.)

The progressive movement of barrel 74 of fixation tool force transfer device 62 causes progressive movement of the plunger 85 of the intermediate syringe 82, as shown in FIGS. 4B, 5B, and 6B, starting from before causing injection (FIG. 4B), in the middle of causing injection (FIG. 5B), and the end of causing injection (FIG. 6B), respectively.

The progressive movement of the plunger 85 of the intermediate syringe 82 causes progressive movement of the plunger 3 of the substance syringe 2, as shown in FIGS. 4C, 5C, and 6C, starting from before causing injection (FIG. 4C), in the middle of causing injection (FIG. 5C), and the end of causing injection (FIG. 6C), respectively.

Alternatively, the intermediate syringe 82 may have no plunger and the force transfer device 62 applies the moving pressure or force (hydraulic, pneumatic, mechanical or electrical) directly to the proximal portion of plunger (plunger rod) 3 of the substance syringe 2, disposed in the barrel 84 of the intermediate syringe 82.

Accordingly, alternatives to the use of a hydraulic or pneumatic syringe as the fixation tool force transfer device 62 include, without limitation, a fluid pump, mechanical cable, electric motor with wire or wireless connections, and the like.

Alternatives to the use of hand holding a fixation tool to simultaneously deliver a substance from an injector held by the other hand, may be any other suitable actuation requiring force, without limitation, such as extension and retraction of a separator within the needle, operation forceps, scissors, other surgical tools.

In some surgical procedures, such as ophthalmic procedures, the user may operate under a microscope with a limited field of view. In such cases, the user does not see his/her operating hands, and it may be difficult to judge the degree of advancement (start, middle, and end) during movement of the member 68 (flanges) of the fixation-tool force transfer device 62. This may make it difficult for the surgeon to know if the delivery of substance is complete, and to know the amount of dose delivered.

Accordingly, as an option, one or more tactile markers 75, may be provided, such as but not limited to, a protrusion, bulge, hole, or texture on fixation-tool force transfer device 62, positioned at a distance equal to the start-to-end of delivery distance of member 68 of the fixation-tool force transfer device 62. The tactile marker 75 serves as tactile indication for the degree of advancement during movement of the member 68 of the fixation-tool force transfer device 62. The one or more tactile markers 75 may be fixed or adjustable.

What is claimed is:

1. A kit comprising:
an injector comprising a syringe, which comprises a plunger that slides in a lumen of a barrel which is coupled to a lumen of a needle;
a fixation tool; and
a coupling assembly that comprises a fixation-tool force transfer device coupled to said fixation tool, wherein said fixation tool force transfer device is coupled to said plunger of said injector such that actuation of said fixation tool force transfer device moves said plunger of said injector.

2. The kit according to claim 1, wherein said fixation-tool force transfer device is not parallel to a shaft of said fixation tool.

3. The kit according to claim 2, wherein an angle between said fixation tool force transfer device and said shaft is fixed.

4. The kit according to claim 2, wherein an angle between said fixation tool force transfer device and said shaft is adjustable.

5. The kit according to claim 1, wherein said fixation-tool force transfer device comprises a plunger slidable in a barrel, wherein movement of said plunger of said fixation-tool force transfer device in said barrel creates a force to move said plunger of said injector.

6. The kit according to claim 5, wherein said plunger of said fixation tool force transfer device is arranged to press against a fluid stored in a barrel of said fixation tool force transfer device.

7. The kit according to claim 1, wherein coupling of said fixation tool force transfer device to said plunger of said injector comprises hydraulic coupling.

8. The kit according to claim 1, wherein coupling of said fixation tool force transfer device to said plunger of said injector comprises pneumatic coupling.

9. The kit according to claim 1, wherein coupling of said fixation tool force transfer device to said plunger of said injector comprises mechanical coupling.

10. The kit according to claim 1, wherein coupling of said fixation tool force transfer device to said plunger of said injector comprises electrical coupling.

11. The kit according to claim 1, wherein said coupling assembly comprises a fluid connection sealingly connected at one end thereof to an exit of a chamber of said fixation tool force transfer device and at an opposite end thereof to a second force transfer device, which is coupled to said plunger of said injector.

12. The kit according to claim 11, wherein said second force transfer device comprises an intermediate syringe, which has a barrel in which said plunger of said injector is received.

13. The kit according to claim 11, wherein said barrel of said second force transfer device is connected to the barrel of the substance syringe of the injector, and movement of the plunger of the fixation-tool force transfer device causes movement of said plunger of said second force transfer device, which causes movement of the plunger of the substance syringe of said injector.
